(19) United States
(12) Patent Application Publication
(10) Pub. No.: US 2006/0257317 A1
(43) Pub. Date: Nov. 16, 2006

(54) Combination therapy in the treatment of cancer

(75) Inventors: Darell D. Bigner, Mebane, NC (US); Michael R. Zalutsky, Chapel Hill, NC (US)

Correspondence Address: DARBY & DARBY P.C., P. O. BOX 5257, NEW YORK, NY, 10150-5257, US

(73) Assignee: Duke University, Durham, NC (US)

(21) Appl. No.: 11/416,633

(22) Filed: May 2, 2006

Related U.S. Application Data
(60) Provisional application No. 60/677,482, filed on May 4, 2005.

(51) Int. Cl. A61K 51/00; A61K 39/395; A61K 31/395

(52) U.S. Cl. 424/1.49; 424/155.1; 514/183

(57) ABSTRACT

Disclosed herein is a method of treating a tumor by administering to the subject a treatment effective amount of a therapeutic antibody and an alkylating agent.

This application claims the benefit of U.S. Provisional Patent Application No. 60/677,482, filed May 4, 2005, which is hereby incorporated by reference in its entirety.

### GOVERNMENT SUPPORT

This invention was made with Government support under grant numbers MO1-RR 30, NS20023, CA11898, CA70164, CA42324, 1P50CA108786-01, 5P20CA96890 and PDT-414 from the National Center for Research Resources General Clinical Research Centers Program, National Institutes of Health and the American Cancer Society. The Government has certain rights to this invention.

### FIELD OF THE INVENTION

The present invention concerns antibody therapy combined with the chemotherapy for the treatment of cancers and tumors in a subject.

### BACKGROUND OF THE INVENTION

The treatment of human cancer with therapeutic antibodies is an emerging approach to this difficult disease. In the United States, two anti-CD20 radiolabeled murine monoclonal antibodies for the treatment of lymphoma have been approved: one under the trade name Zevalin™, produced by IDEC Pharmaceuticals (San Diego, Calif.), and one under the name Bexxar, produced by Corixa Corp. (Seattle, Wash.). There nevertheless remains a need for additional methods for treating cancer, and particularly methods that would aid in increasing specificity and decreasing undesired side-effects of such treatments.

Bigner et al., U.S. Pat. No. 5,624,659, describes methods of treating solid and cystic tumors with monoclonal antibody 81C6. See also D. Bigner et al., “Iodine-131-labeled Anti-tenascin Monoclonal Antibody 81C6 Treatment of Patients with Recurrent Malignant Gliomas: Phase I trial results,” J. Clin. Oncol. 16:2202-2212 (1998). Rizzieri et al., U.S. patent application Ser. No. 10/008,062 (US 2002/0187100 A1, published on Dec. 12, 2002) describes anti-tenascin monoclonal antibody therapy for the treatment of lymphoma. See also D. Rizzieri et al., Blood 104, 642-648 (2004); G. Akabani et al., “Dosimetry and Dose-response Relationships in Newly Diagnosed Patients Treated with Iodine-131-labeled Anti-tenascin Monoclonal Antibody Therapy,” Int. J. Radiat. Oncol. Biol. Phys. 46:947-958 (2000).

### SUMMARY OF THE INVENTION

A first aspect of the invention relates to a method of treating cancer including administering to a subject a treatment effective amount of a therapeutic antibody; and also administering to the subject a treatment effective amount of an alkylating agent.

In one embodiment, the cancer is a solid tumor-based cancer. In a preferred embodiment, the cancer is lymphoma.

In another preferred embodiment, the cancer is brain cancer. In yet another embodiment, the brain cancer is glioblastoma.

In one embodiment, the solid tumor expresses tenascin.

In one embodiment, the therapeutic antibodies specifically bind tenascin.

In one embodiment, the antibody is monoclonal antibody 81C6 or an antibody that binds to the epitope bound by monoclonal antibody 81C6. In a preferred embodiment, the therapeutic antibody is monoclonal antibody 81C6. In another embodiment, the therapeutic antibody is mouse-human chimeric monoclonal antibody 81C6 (ch81C6). In yet another embodiment, the therapeutic antibody is murine monoclonal antibody 81C6 (mu81C6).

In one embodiment, the therapeutic antibodies are coupled to a radionuclide. In another embodiment, the radionuclide is selected from the group consisting of ²²⁷Ac, ²¹¹At, ¹³¹Ba, ⁷⁷Br, ¹⁰⁹Cd, ⁵¹Cr, ⁶⁷Cu, ¹⁶⁵Dy, ¹⁵⁵Eu, ¹⁵³Gd, ¹⁹⁸Au, ¹⁶⁶Ho, ^{113m}In, ^{115m}In, ¹²³I, ¹²⁵I, ¹³¹I, ¹⁸⁹Ir, ¹⁹¹Ir, ¹⁹²Ir, ¹⁹⁴Ir, ⁵²Fe, ⁵⁵Fe, ⁵⁹Fe, ¹⁷⁷Lu, ¹⁰⁹Pd, ³²P, ²²⁶Ra, ¹⁸⁶Re, ¹⁸⁸Re, ¹⁵³Sm, ⁴⁶Sc, ⁴⁷Sc, ⁷²Se, .⁷⁵Se, ¹⁰⁵Ag, ⁸⁹Sr, ³⁵S, ¹⁷⁷Ta, ^{117m}Sn, ¹²¹Sn, ¹⁶⁶Yb, ¹⁶⁹Yb, ⁹⁰Y, ²¹²Bi, ¹¹⁹Sb, ¹⁹⁷Hg, ⁹⁷Ru, ¹⁰⁰Pd, ^{101m}Rh, ²¹²Pb, ⁶⁴Cu, ²²⁵Ac, ²¹³Bi and ¹²⁴I.

In a preferred embodiment, the alkylating agent is temozolomide or an analog, pharmaceutically acceptable salt or prodrug thereof. In yet another embodiment, the temozolomide is administered in a cycle of daily doses for between about 3 to about 7 consecutive days at a daily dose of from between about 50 to about 300 mg/m²/day. In another embodiment, this cycle is repeated every two to five weeks for a total of up to about 10 cycles.

In another embodiment, at least a portion of the tumor is surgically removed before or after or concurrently with administration of the therapeutic antibody.

In one embodiment, the therapeutic antibodies are administered by intracranial injection to the site of the tumor. In another embodiment, the therapeutic antibodies are administered by a single intracranial injection to the site of the tumor.

In a preferred embodiment, the therapeutic antibodies are administered in a dose of between about 40 to about 50 Gy.

In yet another embodiment, the method of treatment further comprises administering the subject external beam radiotherapy to the site of the brain tumor. In a preferred embodiment, external beam radiotherapy is administered at a total dose of between about 30 to about 60 Gy to the site of the brain tumor.

In a preferred embodiment, the therapeutic antibody is monoclonal antibody 81C6, and the alkylating agent is temozolomide or an analog, pharmaceutically acceptable salt or prodrug thereof.

A further aspect of this invention relates to the use of a monoclonal antibody for the preparation of a medicament for carrying out a method of treating cancer in a subject in need thereof, comprising administering to the subject a treatment effective amount of a therapeutic antibody; and also administering to the subject a treatment effective amount of an alkylating agent.

A further aspect of this invention relates to the use of an alkylating agent for the preparation of a medicament for carrying out a method of treating cancer in a subject in need thereof, comprising administering to the subject a treatment effective amount of a therapeutic antibody; and also administering to the subject a treatment effective amount of an alkylating agent.

The foregoing and other objects and aspects of the present invention are explained in detail in the specification set forth below.

### DETAILED DESCRIPTION OF THE EMBODIMENTS OF THE PRESENT INVENTION

The terms “monoclonal antibody 81C6”, “antibody 81C6”, or similar terms encompass both the murine monoclonal antibody 81C6 (mu81C6) and the mouse-human chimeric antibody 81C6 (ch81C6), both of which are described in U.S. Pat. No. 6,624,659. This and all other U.S. patents and U.S. patent applications cited herein are hereby incorporated by reference. Such monoclonal antibodies are produced in accordance with known techniques.

The term “antibodies” as used herein refers to all types of immunoglobulins, including IgG, IgM, IgA, IgD, and IgE. The term “immunoglobulin” includes the subtypes of these immunoglobulins, such as IgG₁, IgG₂, IgG₃, IgG₄, etc. Of these immunoglobulins, IgM and IgG are preferred, and IgG is particularly preferred. The antibodies may be of any species of origin, including (for example) mouse, rat, rabbit, horse, or human, or may be chimeric antibodies. See, e.g., M. Walker et al., Molec. Immunol. 26, 403-11 (1989). The term “antibody” as used herein includes antibody fragments which retain the capability of binding to a target antigen, for example, Fab, F(ab′)₂, and Fv fragments, and the corresponding fragments obtained from antibodies other than IgG. Such fragments are also produced by known techniques.

The term “polyclonal antibody” as used herein refers to multiple immunoglobulins in antiserum produced to an antigen following immunization, and which may recognize and bind to one or more epitopes to that antigen. Polyclonal antibodies used to carry out the present invention can be produced by immunizing a suitable subject of any species of origin, including (for example) mouse, rat, rabbit, goat, sheep, chicken, donkey, horse or human, with an antigen to which a monoclonal antibody to the target binds, collecting immune serum from the animal, and separating the polyclonal antibodies from the immune serum, in accordance with known procedures.

The term “about” or “approximately” as used herein means within an acceptable error range for the particular value as determined by one of ordinary skill in the art, which will depend in part on how the value is measured or determined, i.e., the limitations of the measurement system. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. For example, in the field of radiotherapy, “about 44 Gy” can mean 44 Gy±20% (a range of 35.2 to 52.8 Gy), due to inherent difficulties in measuring radiation absorbed doses (RADs) accurately. Preferably, in practice, “about 44 Gy” means 44 Gy±10% (a range of 39.6 to 48.4 Gy), but this level of accuracy may be difficult to achieve.

The term “pharmaceutically acceptable” as used herein means biologically or pharmacologically compatible for in vivo use in animals or humans, and preferably means approved by a regulatory agency of the Federal or a state government or listed in the U.S. Pharmacopoeia or other generally recognized pharmacopoeia for use in animals, and more particularly in humans.

“Radionuclide” as described herein may be any radionuclide suitable for delivering a therapeutic dosage of radiation to a tumor or cancer cell, including but not limited to ²²⁷Ac, ²¹¹At, ¹³¹Ba, ⁷⁷Br, ¹⁰⁹Cd, ⁵¹Cr, ⁶⁷Cu, ¹⁶⁵Dy, ¹⁵⁵Eu, ¹⁵³Gd, ¹⁹⁸Au, ¹⁶⁶Ho, ^{113m}In, ^{115m}In, ¹²³I, ¹²⁵I, ¹³¹I, ¹⁸⁹Ir, ¹⁹¹Ir, ¹⁹²Ir, ¹⁹⁴ Ir, ⁵²F, ⁵⁵Fe, ⁵⁹Fe, ¹⁷⁷Lu, ¹⁰⁹Pd, ³²P, ²²⁶Ra, ¹⁸⁶Re, ¹⁸⁸Re, ¹⁵³Sm, ⁴⁶Sc, ⁴⁷Sc, ⁷²Se, .⁷⁵Se, ¹⁰⁵Ag, ⁸⁹Sr, ³⁵S, ¹⁷⁷Ta, ^{117m}Sn, ¹²¹Sn, ¹⁶⁶Yb, ¹⁶⁹Yb, ⁹⁰Y, ²¹²Bi, ¹¹⁹Sb, ¹⁹⁷Hg, ⁹⁷Ru, ¹⁰⁰Pd, ^{101m}Rh, ²¹²Pb, ⁶⁴Cu, ²²⁵Ac, ²¹³Bi and ¹²⁴I.

“External beam radiotherapy” is carried out by delivering a beam of high-energy x-rays to the location of the subject's tumor. The beam is generated outside the subject and is targeted at the tumor site. No radioactive sources are placed inside the subject's body.

A “therapeutically effective amount” as used herein means the amount of a compound that, when administered to a subject for treating a state, disorder or condition is sufficient to effect a treatment (as defined below). The “therapeutically effective amount” will vary depending on the compound, the disease and its severity and the age, weight, physical condition and responsiveness of the subject to be treated. According to the present invention, in one embodiment, a therapeutically effective amount of radio-labeled antibody is an amount effective to treat various cancers. In another embodiment, a therapeutically effective amount of unlabeled antibody is an amount effective to block the binding of radio-labeled antibody to healthy, non-target tissue.

“Treat” as used herein refers to any type of treatment or prevention that imparts a benefit to a subject afflicted with a disease or at risk of developing the disease, including improvement in the condition of the subject (e.g., in one or more symptoms), delay in the progression of the disease, delay the onset of symptoms or slow the progression of symptoms, etc. As such, the term “treatment” also includes prophylactic treatment of the subject to prevent the onset of symptoms. As used herein, “treatment” and “prevention” are not necessarily meant to imply cure or complete abolition of symptoms.”

“Treatment effective amount” as used herein means an amount of the antibody sufficient to produce a desirable effect upon a subject inflicted with cancer, including improvement in the condition of the subject (e.g., in one or more symptoms), delay in the progression of the disease, etc.

A “subject” or “subject in need” is a human or non-human mammal in need of radioimmunotherapy” (RIT), chemoimmunotherapy, cytotoxic immunotherapy or some other therapeutic method according to the present invention.

A “surgically created resection cavity” (“SCRC”) is a cavity in the brain that is surgically created during the removal of a brain tumor, such as a glioblastoma multiforme (“GBM”). A “margin” is a region of parenchyma or brain tissue surrounding the SCRC and may be expressed in terms of a distance from the SCRC/parenchyma interface, or outside edge of the SCRC.

A “region of interest” (“ROI”) as used herein is a defined region of tissue in or near the SCRC. An ROI may be a region that is located at the margin (or outside edge) of the SCRC. “Parenchyma,” or “parenchymal tissue” as used herein consists of tissue composed of functional cells. Parenchymal cells are much less tolerant of a degraded environment, e.g., an SCRC, than are structural cells, or mesenchymal tissue. An SCRC “interface” as used herein describes the border between the SCRC and surrounding healthy tissue.

A “residence time” as used herein is a measurement of how long a radionuclide is retained in the body. A “whole-body clearance rate” is a total body residence time.

An “S-value” as used herein is a value that describes the absorbed dose to a specific target region from radiation emitting from another source. S-values may be derived using Monte Carlo methods and MIRD phantom, according to calculations known by those of skill in the art. S-values are dependent on the size of the surgically created resection cavity (SCRC), which may range from below about 2 cm³ (an S-value of about 9.60E-3 Gy hr mCi⁻¹), to about 60 cm³ (an S-value of about (2.34E-3 Gy hr mCi⁻¹), or beyond.

An “absorbed dose” as used herein is the radiation energy (or radioactivity) absorbed (or “deposited”) in a region of interest or other material per unit of mass of the material. This is different from the “administered” or “therapeutic” or “radioimmunotherapy” (RIT) dose, which is the total amount of radioactivity administered to a subject. The absorbed dose refers only to the amount of radiation energy (or radioactivity) that has been administered and absorbed (or “deposited”) into tissue. “Absorbed dose” is alternatively known as “Radiation Absorbed Dose,” or “RAD.” If the absorbed dose or RIT dose is determined before employing the dosimetric methods according to this invention to estimate an RIT dose, the absorbed dose or RAD is called a “predetermined absorbed dose” or a “predetermined RAD.” The predetermined RAD may be a predetermined optimal RAD based on experimental data. A predetermined optimal RAD may be determined by any means accepted in the field of cancer or disease therapy, including experimental trials where the safety and efficacy of a particular radiotherapeutic agent can be determined. For example, an optimal RAD can be determined based on toxicity and clinical outcome in an observed group of subjects. In one embodiment, the predetermined optimal RAD is about 44 Gy.

A “radioimmunotherapy dose” (“RIT dose”) as used herein is the dose of an RIT agent to be delivered for therapeutic purposes. A therapeutic RIT dose is calculated to achieve a predetermined radiation absorbed dose (RAD).

A “targeting moiety” as used herein is any moiety that is able to bind to, i.e., a “binding partner of,” the intended target of the therapy, and deliver an amount of a radio-label (radiotherapeutic agent), chemotherapeutic agent, cytotoxic agent, or other therapeutic agent known in the art. For instance, a targeting moiety may be a receptor ligand in instances when the target is a cellular receptor. Preferably, the therapeutic agent is an antibody, e.g., 81C6 monoclonal antibody. When the targeting moiety is an antibody and the therapeutic agent is a radio-label, the complex may be called a radioimmunotherapy (RIT) dose.

A “dosimetric dose” as described herein is a small dose (“sub-therapeutic”) used to calculate a RIT dose to be administered in the future. A number of dosimetric doses are administered in increasing amounts, after which a series of dose-response analyses are performed and the desired RIT dose is determined, based on a predetermined absorbed dose.

“Extracellular stromal constituent” as used herein refers to a compound specific to the extracellular (as opposed to the cellular or cell surface) space, including the glycocalyx, the extracellular matrix, and the basal lamina. Examples of extracellular stromal constituents include but are not limited to fibrinogen, fibronectin, collagen, laminin, proteoglycan, tenascin, entactin, and thrombospondin. If the cellular constituent comprises tumor or cancer cells, the extracellular stromal constituent is the extracellular stromal constituent “of the tumor.” A blocking antibody that binds tenascin in the extracellular stromal constituent will bind to tenascin molecules in the extracellular stromal constituent of both normal and tumorous tissue.

“Chemotherapeutic agent” as used herein includes but is not limited to methotrexate, daunomycin, mitomycin, cisplatin, vincristine, epirubicin, fluorouracil, verapamil, cyclophosphamide, cytosine arabinoside, aminopterin, bleomycin, mitomycin C, democolcine, etoposide, mithramycin, chlorambucil, melphalan, daunorubicin, doxorubicin, tamosifen, paclitaxel, vincristin, vinblastine, camptothecin, actinomycin D, and cytarabine

“Cytotoxic agent” as used herein includes but is not limited to ricin (or more particularly the ricin A chain), aclacinomycin, diphtheria toxin. Monensin, Verrucarin A, Abrin, Vinca alkaloids, Tricothecenes, and Pseudomonas exotoxin A.

“Radioimmunotherapy” or “RIT” as used herein refers to therapy using an antibody conjugated to a radionuclide (or radio-label).

“Gy” as used herein refers to a unit for a specific absorbed dose of radiation equal to 100 Rads. Gy is the abbreviation for “Gray.”

“Chemoimmunotherapy” as used herein refers to therapy using an antibody conjugated to a chemotherapeutic agent.

“Cytotoxic immunotherapy” as used herein refers to therapy using an antibody conjugated to a cytotoxic agent.

A “therapeutic antibody” as used herein is an antibody that is conjugated to a radionuclide (or “radio-label”), a chemotherapeutic agent, or a cytotoxic agent. When the therapeutic antibody is conjugated to a radionuclide (or radio-label), it is known as an RIT agent or an RIT antibody.

An “alkylating agent” as used herein is a compound that has the ability to add alkyl groups (alkyl groups are compounds containing only carbon and hydrogen and have the general formula CₙH₂ₙ₊₁, e.g., a methyl group (CH₃)) to electronegative groups, e.g., nucleic acids, under conditions present in cells. They stop tumor growth by cross-linking guanine nucleotides in DNA double-helix strands. This makes the DNA strands unable to uncoil and separate. As this is necessary in DNA replication, the cells can no longer divide. Because cancer cells generally divide more rapidly than do healthy cells, they are more sensitive to DNA damage. alkylating agents are used clinically to treat a variety of tumors. One example of an alkylating agent is temozolomide, which may be used to treat a variety of cancers, including the cancers that are the subject of the treatment methods herein. “Temozolomide” as it is used herein refers to temozolomide and all anlogs, pharmaceutically acceptable salts and prodrugs thereof.

A “prodrug” as used herein is a pharmacological drug which is administered in an inactive (or significantly less active) form. Once administered, the prodrug is metabolized in the body in vivo into the active compound.

1. Subjects.

Subjects in need of treatment by the methods described herein include subjects afflicted with lymphoma, as well as subjects afflicted with solid tumors or cancers such as lung, colon, breast, brain, liver, prostate, spleen, muscle, ovary, pancreas, skin (including melanoma) etc.

Subjects to be treated by the methods of the invention particularly include subjects afflicted with a tumor expressing tenascin, including gliomas, fibrosarcomas, osteosarcomas, melanoma, Wilms tumor, colon carcinoma, mammary and lung carcinomas, and squamous carcinomas.

Subjects to be treated by the present invention most particularly include subjects afflicted with brain tumors or cancers, such as glioblastomas, particularly glioblastoma multiforme, and cystic astrocytoma.

The present invention is primarily concerned with the treatment of human subjects, including male and female subjects and neonatal, infant, juvenile, adolescent, adult, and geriatric subjects, but the invention may also be carried out on animal subjects, particularly mammalian subjects such as mice, rats, dogs, cats, livestock and horses for veterinary purposes, and for drug screening and drug development purposes.

2. Antibodies.

Antibodies that bind to extracellular stromal constituents of cancers and tumors are known and described in, for example, U.S. Pat. Nos. 6,783,760 and 6,749,853.

Antibodies employed in carrying out the present invention may be those which bind to tenascin. Particularly preferred anti-tenascin monoclonal antibodies are monoclonal antibody 81C6 (MAb 81C6) and antibodies that bind to the epitope bound by monoclonal antibody 81C6 (i.e., antibodies that cross-react with, or block the binding of, monoclonal antibody 81C6). Antibodies can be produced by any suitable technique, such as in nude mouse ascites, hollow fiber culture, suspension culture, etc. The monoclonal antibody 81C6 is in one embodiment a murine IgG2b monoclonal antibody raised from a hybridoma fusion following immunization of BALB/c mice with the glial fibrillary acidic protein (GFAP)-expressing permanent human glioma line U-251 MG, as known and described in M. Bourdon et al., Cancer Res. 43, 2796 (1983) (mu81C6).

Particularly preferred for carrying out the present invention is a mouse-human chimeric monoclonal antibody 81C6 (ch81C6), as described in U.S. Pat. No. 5,624,659 to Bigner and Zalutsky, or murine monoclonal antibody 81C6 as described in M. Bourdon et al., supra.

Antibodies for use in the present invention specifically bind to tenascin with a relatively high binding affinity, for example, with a dissociation constant of about 10⁻⁴ to 10⁻¹³. In embodiments of the invention, the dissociation constant of the antibody-tenascin complex is at least 10⁻⁴, preferably at least 10⁻⁶, and more preferably at least 10⁻⁹.

Blocking antibodies that may be used in conjunction with administration of therapeutic antibodies according to the present invention are, in general, not coupled or conjugated to any therapeutic agent, while therapeutic antibodies used to carry out the present invention are, in general, coupled or conjugated to a therapeutic agent. Thus blocking antibodies are not themselves therapeutically active in treating cancer in the methods described herein.

Antibodies used for therapy (i.e., in a method of combating cancer) may be polyclonal or monoclonal antibodies per se or monoclonal antibodies coupled to a therapeutic agent. Such antibodies are sometimes referred to herein as therapeutic antibodies.

Any therapeutic agent conventionally coupled to a monoclonal antibody may be employed, including (but not limited to) radionuclides, cytotoxic agents, and chemotherapeutic agents. See generally Monoclonal Antibodies and Cancer Therapy (R. Reisfeld and S. Sell Eds. 1985) (Alan R. Liss Inc. N.Y.). Therapeutic agents such as radionuclides, cytotoxic agents and chemotherapeutic agents are known and described in U.S. Pat. Nos. 6,787,153; 6,783,760; 6,676,924; 6,455,026; and 6,274,118.

Therapeutic agents may be coupled to the antibody by direct means or indirect means (e.g., via a chelator) by any suitable technique, including but not limited to those described in U.S. Pat. Nos. 6,787,153; 6,783,760; 6,676,924; 6,455,026; and 6,274,118. Therapeutic agents may be coupled or conjugated to the antibody by the Iodogen method or with N-succinimidyl-3-(tri-n-butylstanyl)benzoate (the “ATE method”), as will be apparent to those skilled in the art. See, e.g., M. Zalutsky and A. Narula, Appl. Radiat. Isot. 38, 1051 (1987).

It will be appreciated that monoclonal antibodies as used herein incorporate those portions of the constant region of an antibody necessary to evoke the useful immunological response in the subject being affected.

3. Antibody Formulations.

The blocking antibodies and therapeutic antibodies will each generally be mixed, prior to administration, with a non-toxic, pharmaceutically acceptable carrier substance (e.g. normal saline or phosphate-buffered saline), and will be administered using any medically appropriate procedure, e.g., parenteral administration (e.g., injection) such as by intravenous or intra-arterial injection.

The blocking antibodies and therapeutic antibodies compounds described above may be formulated for administration in a pharmaceutical carrier in accordance with known techniques. See, e.g., Remington, The Science And Practice of Pharmacy (9^{th} Ed. 1995). In the manufacture of a pharmaceutical formulation according to the invention, the active compound (including the physiologically acceptable salts thereof) is typically admixed with, inter alia, an acceptable carrier. The carrier must, of course, be acceptable in the sense of being compatible with any other ingredients in the formulation and must not be deleterious to the subject. The carrier may be a liquid and is preferably formulated with the compound as a unit-dose formulation which may contain from 0.01 or 0.5% to 95% or 99% by weight of the active compound.

As discussed further below, the therapeutic antibodies may optionally be administered in conjunction with other, different, active compounds useful in the treatment of the disorders or conditions described herein (e.g., chemotherapeutics). The other compounds may be administered concurrently. As used herein, the word “concurrently” means sufficiently close in time to produce a combined effect (that is, concurrently may be simultaneously, or it may be two or more administrations occurring before or after each other).

Formulations of the present invention suitable for parenteral administration comprise sterile aqueous and non-aqueous injection solutions of the active compound, which preparations are preferably isotonic with the blood of the intended recipient. These preparations may contain anti-oxidants, buffers, bacteriostats and solutes which render the formulation isotonic with the blood of the intended recipient.

Blocking and therapeutic antibodies may be provided in lyophilized form in a sterile aseptic container or may be provided in a pharmaceutical formulation in combination with a pharmaceutically acceptable carrier, such as sterile pyrogen-free water or sterile pyrogen-free physiological saline solution.

4. Examples of Tumors, Cancers, and Neoplastic Tissue.

Examples of tumors, cancers, and neoplastic tissue that can be treated according to the present invention include, but are not limited to, malignant disorders such as breast cancers; osteosarcomas; angiosarcomas; fibrosarcomas and other sarcomas; leukemias; lymphomas (Hodgkin's lymphoma and Non-Hodgkin's lymphoma), and other blood cancers; myelodysplasia, myeloproliferative disorders; sinus tumors; ovarian, uretal, bladder, prostate and other genitourinary cancers; colon, esophageal and stomach cancers and other gastrointestinal cancers; lung cancers; myelomas; pancreatic cancers; liver cancers; kidney cancers; endocrine cancers; skin cancers; and brain or central and peripheral nervous system tumors, malignant or benign, including gliomas and neuroblastomas.

5. Dosimetry Studies.

The amount of therapeutic antibody administered is, in some embodiments, determined through a dosimetry study prior to administration of a therapeutic dosage. For example, a method for dosimetry estimation for a region of interest surrounding a resection cavity in a subject, may be carried out by: determining a size of the resection cavity; determining a residence time based on the size of the resection cavity and detected radiation from the region of interest at a plurality of times subsequent to administering a dosimetric Radio-immunotherapy (“RIT”) dose; and calculating an administered therapeutic RIT dose based on the residence time, the size of the resection cavity, and a predetermined absorbed dose (e.g., an optimal absorbed dose based on experimental data, in some embodiments about 44 Gy).

The dosimetry method may include performing whole-body scintigraphy to detect radiation from the region of interest (e.g., wherein the scintigraphy is performed at a first time that is substantially the same time as administering the dosimetric RIT dose, at a second time that is about twenty-four hours subsequent to the first time, and at a third time that is about forty-eight hours subsequent to the first time).

The dosimetry method may include performing magnetic resonance imaging to determine the size of the resection cavity.

In some embodiments, the region of interest is a region of parenchyma up to about one or two centimeters from the margin of the resection cavity.

The administered therapeutic RIT dose may, for example, be calculated based on the formula:

$${A}_{0} = \frac{{D}_{SCRC}}{S ( {B}_{2 \text{-} cm} ← SCRC ) {τ}_{SCRC}}$$

where D_{SCRC} is the predetermined absorbed dose, S(B_{2-cm}←SCRC) is an estimated S-value based on the size of the resection cavity in Gy hr mCi⁻¹, and τ_{SCRC} is the resection cavity residence time.
6. Antibody Administration.

The blocking antibodies and therapeutic antibodies may be administered by any medically appropriate procedure, e.g., normal intravenous or intra-arterial administration, injection into the cerebrospinal fluid). In certain cases, intradermal, intracavity, intrathecal or direct administration to the tumor or to an artery supplying the tumor is advantageous.

Dosage of the blocking antibody will depend, among other things, on the condition of the subject, the particular category or type of cancer being treated, the route of administration, the nature of the therapeutic agent employed, and the sensitivity of the tumor to the particular therapeutic agent. For example, the dosage will typically be about 1 to 10 micrograms per kilogram subject body weight. The specific dosage of the antibody is not critical, as long as it is effective to result in some beneficial effects in some individuals within an affected population. In general, the dosage may be as low as about 0.05, 0.1, 0.5, 1, 5, 10, 20 or 50 micrograms per kilogram subject body weight, or lower, and as high as about 5, 10, 20, 50, 75 or 100 micrograms per kilogram subject body weight, or even higher.

Dosage of the therapeutic antibody will likewise depend, among other things, the condition of the subject, the particular category or type of cancer being treated, the route of administration, the nature of the therapeutic agent employed, and the sensitivity of the tumor to the particular therapeutic agent. For example, the dosage will typically be about 1 to 10 micrograms per kilogram subject body weight. The specific dosage of the antibody is not critical, as long as it is effective to result in some beneficial effects in some individuals within an affected population. In general, the dosage may be as low as about 0.05, 0.1, 0.5, 1, 5, 10, 20 or 50 micrograms per kilogram subject body weight, or lower, and as high as about 5, 10, 20, 50, 75 or 100 micrograms per kilogram subject body weight, or even higher.

In another example, where the therapeutic agent is ¹³¹I, the dosage to the subject will typically be from 10 mCi to 100, 300 or even 500 mCi. Stated otherwise, where the therapeutic agent is ¹³¹I, the dosage to the subject will typically be from 5,000 Rads (50 Gy) to 100,000 Rads (1000 Gy) (preferably at least 13,000 Rads (130 Gy), or even at least 50,000 Rads (500 Gy). Doses for other radionuclides are typically selected so that the tumoricidal dose will be equivalent to the foregoing range for ¹³¹I, even though the amount of radiation may be different. For example, only a few millicuries of ²¹¹At may be required to deliver a radiation dose to tumors the equivalent of that delivered by 100 millicuries of ¹³¹I.

The therapeutic antibody may be administered by any suitable means including intravenous injection and injection into the tumor. Where the tumor or a portion thereof has been previously surgically removed the antibody may be administered into the site of the tumor (and particularly into an enclosed cavity or “resection cavity” at the site of the tumor) by direct injection or through a pre-implanted reservoir.

In a preferred embodiment, the therapeutic antibodies are administered in a dose of 30 to 60 Gy, more preferably 40 to 50 Gy, still more preferably 40 to 48 Gy, and most preferably 44 Gy, with the dose preferably determined by means of a dosimetry study as described above.

7. Alkylating Agents.

Alkylating agents useful for carrying out the present invention include, but are not limited to, 1,3-bis(2-chloroethyl)-1-nitrosourea (BCNU) and tetrazine derivatives, particularly [3H]imidazo[5,1-d]1,2,3,5-tetrazin-4-one derivatives such as temozolomide and analogs thereof, including pharmaceutically acceptable salts and prodrugs thereof. Such compounds are known. See, e.g., U.S. Pat. Nos. 6,096,724; 6,844,434; and 5,260,291.

Examples of alkylating agents useful for carrying out the present invention include [3H]-imidazo[5,1-d]-1,2,3,5-tetrazin-4-ones alkylating agents, particularly those of the general formula: 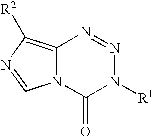
wherein R¹ represents a hydrogen atom, or a straight- or branched-chain alkyl, alkenyl or alkynyl group containing up to 6 carbon atoms, each such group being unsubstituted or substituted by from one to three substituents selected from halogen (i.e. bromine, iodine or, preferably, chlorine or fluorine) atoms, straight- or branched-chain alkoxy, (e.g. methoxy), alkylthio, alkylsullihinyl, or alkylsulphonyl groups containing up to 4 carbon atoms, and optionally substituted phenyl groups. Alternatively, R¹ represents a cycloalkyl group, and R² represents a carbamoyl group which may carry on the nitrogen atom one or two groups selected from straight- and branched-chain alkyl and alkenyl groups, each containing up to 4 carbon atoms, and cycloalkyl groups, e.g. a methylcarbamoyl or dimethylcarbamoyl group. When the symbol R¹ represents an alkyl, alkenyl or alkynyl group substituted by two or three halogen atoms, the aforementioned halogen atoms may be the same or different. When the symbol R¹ represents an alkyl, alkenyl or alkynyl group substituted by one, two or three optionally substituted phenyl groups, the optional substituents on the phenyl radical(s) may be selected from, for example, alkoxy and alkyl groups containing up to 4 carbon atoms (e.g. methoxy and/or methyl group(s)) and the nitro group; the symbol R¹ may represent, for example, a benzyl or p-methoxybenzyl group. Cycloalkyl groups within the definitions of symbols R¹ and R² contain 3 to 8, preferably 6, carbon atoms. The compounds may be provided as salts or prodrugs, particularly alkali metal salts when R¹ is H. See, e.g., U.S. Pat. No. 5,260,291.

Temozolomide, in 5 mg, 20 mg, 100 mg, and 250 mg oral dosage form as capsules, is commercially available as TEMODAR® from Schering Corporation, Kenilworth N.J. 07033 USA.

In a preferred embodiment, the alkylating agent is administered in a cycle of daily doses for 3, 4, 5, 6 or 7 consecutive days. A suitable daily dose may be from 50, 100 or 150 mg/m²/dose, up to 200, 250 or 300 mg/m²/dose. This cycle may be repeated, e.g. every two, three, four or five weeks, for up to a total of 6, 8 or 10 cycles. The first dose in the first cycle of alkylating agent may be administered at any suitable point in time. In some embodiments the first dose of alkylating agent is administered up to two or four weeks before administration of the therapeutic antibody; in some embodiments the first dose of alkylating agent is administered at least two, four or six weeks following the administration of the therapeutic antibody. In another embodiment, the agent can be administered concomitantly with the antibody therapy. Additional schedules of administration may be included where additional therapeutic treatments such as external beam radiotherapy are also applied to the subject.

8. External Beam Radiotherapy.

Optionally, but in some embodiments preferably, the subject also receives external beam radiotherapy. For example, external beam radiotherapy is particularly preferred for brain tumors such as glioblastoma. External beam radiotherapy is known and can be carried out in accordance with known techniques. The beam can be generated by any suitable means, including medical linear accelerators and Cobalt 60 external beam units. The radiation source can be mounted in a gantry that rotates around the subject so that a target area within the subject is irradiated from different directions. Before irradiation the treatment is typically planned on a computer using algorithms that simulate the radiation beams and allow the medical personnel to design the beam therapy. Numerous variations of external beam therapy that can be used to carry out the present invention will be readily apparent to those skilled in the art. See, e.g., U.S. Pat. Nos. 6,882,702; 6,879,659; 6,865,253; 6,863,704; 6,826,254; 6,792,074; 6,714,620; and 5,528,650.

External beam therapy is preferably administered in a series of sessions in accordance with known techniques, with the sessions preferably beginning two to four weeks after administration of the therapeutic antibody. For example, the external beam radiotherapy may be administered 3, 4, 5, 6 or 7 days a week, over a period of four, five, six or seven weeks, at a daily dose of 0.5 or 1 Gy, up to 2 or 3 Gy, until the total desired dose (e.g., 30 or 40 Gy, up to 50 or 60 Gy) is administered.

The delivered dose may be to an area including a margin of normal tissue (e.g., a 1, 2 or 3 cm margin in all directions) around the tumor, or—where the tumor or a portion thereof has previously been surgically removed—around the site of the tumor.

Where external beam radiotherapy is employed, the subject may receive an additional schedule of alkylating agent administration, different from that described above, at a somewhat lower dose, during the course of the radiotherapy. For example, the subject may receive daily doses of alkylating agent in an amount of from 25 or 50 mg/m²/dose up to 100 or 125 mg/m²/dose daily during the course of the external beam therapy.

### EXAMPLES

The present invention will be better understood by reference to the following Examples, which are provided as exemplary of the invention, and not by way of limitation.

### Example 1

### Production of 81C6

The production of murine monoclonal antibody 81C6 is known. See, e.g., M. Bourdon et al., Cancer Res. 43, 2796 (1983). Monoclonal antibody 81C6 can be produced by any suitable technique, such as in nude mouse ascites, hollow fiber culture, suspension culture, etc.

In one embodiment, nude mouse produced ascites fluid containing immunoglobulin is ultracentrifuged at 125000×g for 45 minutes. Supernatant is filter sterilized through a 0.22 Millistak (Millipore) filter. Immunoglobulin is purified from ascites by passing over a protein-A Sepharose column which is sterilized by flushing column with 10 column volumes of 4 M guanidine-HCl. After rinsing the column with 10 column volumes of Tris-NaCl buffer (10 mM Tris in 0.9% NaCl, pH 8.0), ascites is passed through the protein-A column. The column is rinsed with pH 8.0 Tris-NaCl buffer and bound immunoglobulin eluted with pH 3.0 glycine HCl buffer (0.55 M glycine, 0.85% NaCl and 10 mM HCl.). Fractions are collected and immediately neutralized with 0.5 milliliters of 1 M Tris buffer, pH 8.0. Absorbance is read at 280 nm in a flow through spectrophotometer and the fractions containing immunoglobulin are pooled. Purity of pooled immunoglobulin is checked by gel filtration on a HPLC TSK-3000 column and then dialyzed overnight against 20 volumes of 75 mM Tris-acetate buffer (pH 6.0) in a 50,000 molecular weight cut off dialysis tubing. Forty micron size polyethyleneimine (PEI) is obtained from JT Baker Company in bulk and the appropriate size column is packed for the amount of immunoglobulin to be bound. One gram of dry PEI or ABx will bind 200 mg of immunoglobulin under ideal conditions. Stainless steel columns are heated to 210° C. for four hours prior to packing to remove any endotoxins. PEI columns are sterilized by flushing with 10 column volumes of 4 M guanidine. Columns are then equilibrated by flushing with 20 to 30 column volumes of water and 75 mM Tris acetate buffer (pH 6.0). Dialyzed immunoglobulin is injected onto PEI column. The column is rinsed with equilibration buffer until the 280 nanometer (nm) absorbance baseline returns to zero. Elution of bound antibody from the PEI column is accomplished by running a 60 minute linear gradient from 0%/100% 2 M Na Acetate buffer (pH 6.8) and 1 milliliter fractions are collected. Absorbance of eluent is monitored at 280 nm. Tubes containing immunoglobulin are pooled and dialyzed exhaustively against 115 mM phosphate buffer, pH 7.4. Endotoxin is removed by passing antibody over an ActiClean Etox column (Sterogene; Carlsbad, Calif.) and then dialyzed against 115 mM phosphate buffer pH 7.4. After dialysis, protein concentration is determined and adjusted to 15 to 16 mg/ml for Rx dose ampoules and 5 mg/ml/ampoule for dose quantitation studies. Aliquots are made into sterile and pyrogen free vials by injecting solution directly into vials through a 0.22 micron Millpore filter. Protein concentration is determined after filtering to make sure no protein is lost during filtration. Quality controls that are run on the batch are Sterility, Limulus Amebocyte Lysate (LAL) assay for endotoxins, gel filtration HPLC on a Super TSK 3000 column for both ¹³¹I-labeled and unlabelled mu81C6, and PAGE (Polyacrylamide gel electrophoresis). The immunoreactive fraction of ¹³¹I-labeled mu81C6 is checked by the Lindmo Method, according to Lindmo et al., “Determination of the Immunoreactive Fraction of Radiolabeled Monoclonal Antibodies by Linear Extrapolation to Binding at Infinite Antigen Excess,” J. Immunol. Methods 72: 77-89 (1994).

### Example 2

### Production of ¹³¹I-Labeled 81C6

Sodium Iodide ¹³¹I in 0.1 N Sodium Hydroxide solution at a specific concentration of approximately 1000 mCi/ml is purchased from Perkin Elmer Life Sciences (Boston, Mass.). The 81C6 antibody is prepared as described above. All procedures are accomplished in a Baker vertical laminar flow hood with 100% venting through charcoal filters to the outside. Aseptic technique is employed throughout. All measurements of radioactivity are made with a Capintec (Ramsey, N.J.) dose calibrator. Two mg aliquots of the antibody are incubated for 10 minutes in multiple 1 ml glass vials which each have 10 micrograms iodogen (a coupling reagent) dried on the inner surface. Each vial also contains 0.05 M phosphate buffered saline pH 7.2-7.4 (PBS), and 25 mCi of ¹³¹I (approximately 25 μL ¹³¹I solution) in a total volume of 0.250 ml at pH 7.2-7.4. The vial contents are then pooled and the vials are rinsed twice with 0.15 ml PBS and are pooled into a 15 ml sterile plastic culture tube. Purification is done on a Sephadex G-25 (Sigma, St. Louis, Mo.) column pre-treated with 0.100 ml of 5% human serum albumin, USP, to saturate nonspecific protein binding sites in the resin, and eluted with PBS. Twenty 0.5 ml fractions are collected from the column into sterile 3 ml plastic culture tubes. The contents of the fraction tubes containing the greatest amount of ¹³¹I associated with the peak corresponding to the 81C6 antibody-containing fraction are drawn into a sterile plastic 10 cc disposable syringe with a 3.5 inch spinal needle attached and are pooled in another 15 ml plastic culture tube. Each of the fraction tubes is then rinsed twice with a solution of 1% human serum albumin, USP, in PBS, pH 7.2-7.4. After the second rinse, the human serum albumin solution is mixed with the pooled fractions and all fractions are drawn into the 10 cc syringe—the final volume is typically 3-5 ml. Sterilization is accomplished by replacing the spinal needle with a sterile Millipore 0.22 micron membrane filter (Millex GV; Millipore) attached to a 20 gauge needle and injecting the solution into a sterile 10 ml evacuated vial (the final vial). The amount of Antibody 81C6 protein in the vial is estimated by first estimating the Specific Activity (SA), assuming 100% recovery of protein added to the reaction into the pooled active fractions. Then, the measured activity in the final vial (in mCi) is divided by the SA, and a value is calculated for the total amount of protein in the vial. The total amount of antibody protein in the subject dose is prescribed. By dividing the total activity (mCi) in the vial by the subject dose activity (mCi) and then multiplying this ratio by the total mg of protein prescribed, an estimate of the total amount of protein required in the vial is calculated. By subtracting the amount of protein already in the vial from the total amount of protein required in the vial yields an estimate of nonradioactive “cold” Antibody 81C6 needs to be added to the vial. The final product has the prescribed activity of 131, (mCi) and Antibody 81C6 protein (mg) in 0.05 M PBS and approximately 0.5% human serum albumin, USP. USP Sterility testing, LEL testing for bacterial endotoxin, radionuclide purity testing, radiochemical purity testing by HPLC and radioimmunoreactivity testing is performed. After all quality control (QC) is completed, the subject dose is drawn up in the into a 10 cc or 20 cc sterile disposable plastic syringe; the syringe is sealed with a sterile syringe cap, appropriately labeled and witnessed, shielded with lead and sent to Nuclear Medicine for administration to the subject.

### Example 3

### ¹³¹I-81C6-Temozolomide Combination Therapy for the Treatment of Glioblastoma Multiforme (GBM)

### Route of Administration

¹³¹I-81C6 can be administered into a cystic cavity created by surgical resection of glioblastoma multiforme via an indwelling intracranial resection cavity Rickham catheter placed at the time of tumor resection. It will be understood by those of ordinary skill in the art that other routes of administration may be possible.

### Subjects for Treatment

Suitable subjects for treatment will be those subjects with a confirmed histologic diagnosis of a newly diagnosed and previously untreated supratentorial glioblastoma multiforme (GBM). Reactivity of neoplastic cells with tenascin may be demonstrated by immunohistology with either a polyclonal rabbit antibody or the monoclonal mouse antibody. Subjects who have received any therapy other than surgical resection may not eligible. Other therapies, which optionally may be performed in conjunction with ¹³¹I-81C6-Temozolomide combination therapy may include radiotherapy, chemotherapy, immunotherapy, or any other experimental therapy used to treat the GBM.

The subject may be a candidate for surgical resection. In this case, a contrast-enhanced CT or magnetic resonance imaging (MRI) should be obtained less than 72 hours after surgery. An interval of at least 2 weeks between prior surgical resection and ¹³¹I-81C6 administration may be necessary.

The following baseline blood values should be determined prior to ¹³¹I-81C6 administration: hemoglobin level; absolute neutrophil count; platelet count; creatinine level; bilirubin level, and serum glutamic oxaloacetic transaminase level. Certain baseline blood values for these blood constituents may have to be established before ¹³¹I-81C6 administration can commence.

In addition, a 99 mTc-DTPA flow study may be used to demonstrate adequate placement of the Rickham catheter in the SCRC and lack of communication between the SCRC and the CSF space. Also, a dosimetry study may be performed. Dosimetry refers to the accurate measurement of dosages. If the doses in question are radioactivity doses, dosimetry refers to the accurate measurement of the amount of radiation energy in a tissue. This is especially critical when using radiation to treat a subject for a disease or cancer. Thus, using too much radiation is very toxic to the subject, while using too little is ineffective as therapy. Dosimetry provides for an accurate determination of a safe and effective RIT dose (the amount of administered radioactivity).

It will be understood by those of ordinary skill in the art that subjects with cancers other than GBM, including, inter alia, lymphomas, may be treated according to the methods disclosed herein.

### Therapy Method

Preferably, treatment begins with ¹³¹I-81C6 administration. It will be understood by those of ordinary skill in the art that the 81C6 antibody being used may be from human, mouse, or any suitable species, and may be a chimeric antibody. In addition, antibodies other than 81C6 that bind tenascin may be used. It will be further understood by those of ordinary skill in the art that antibodies other than those that recognize tenascin may be used in accordance with the method according to this invention. Preferably, the antibody used will be labeled with ¹³¹I. However, other radionuclides, including, but not limited to, ²²⁷Ac, ²¹¹At, ¹³¹Ba, ⁷⁷Br, ¹⁰⁹Cd, ⁵¹Cr, ⁶⁷Cu, ¹⁶⁵Dy, ¹⁵⁵Eu, ¹⁵³Gd, ¹⁹⁸Au, ¹⁶⁶Ho, ^{113m}In, ^{115m}In, ¹²³I, ¹²⁵I, ¹³¹I, ¹⁸⁹Ir, ¹⁹¹Ir, ¹⁹²Ir, ¹⁹⁴Ir, ⁵²Fe, ⁵⁵Fe, ⁵⁹Fe, ¹⁷⁷Lu, ¹⁰⁹Pd, ³²P, ²²⁶Ra, ¹⁸⁶Re, ¹⁸⁸Re, ¹⁵³Sm, ⁴⁶Sc, ⁴⁷Sc, ⁷²Se, .⁷⁵Se, ¹⁰⁵Ag, ⁸⁹Sr, ³⁵S, ¹⁷⁷Ta, ^{117m}Sn, ¹²¹Sn, ¹⁶⁶Yb, ¹⁶⁹Yb, ⁹⁰Y, ²¹²Bi, ¹¹⁹Sb, ¹⁹⁷Hg, ⁹⁷Ru, ¹⁰⁰Pd, ^{101m}Rh, ²¹²Pb, ⁶⁴Cu, ²²⁵Ac, ²¹³Bi and ¹²⁴I may be used. In addition, the antibody used may be coupled to other suitable therapeutic agents, including, inter alia, chemotherapeutic agents. Furthermore, the antibody used may be coupled to more than one therapeutic agent.

### External Beam Radiotherapy (XRT) Administration

Optionally, subjects will undergo external beam radiotherapy as part of the treatment. Preferably, external beam radiotherapy would take place approximately 4 weeks following administration of ¹³¹I-81C6 antibodies. However, it will be understood by a person of ordinary skill in the art that the timing of different aspects of the therapy according to the present invention may vary according to the subject and cancer being treated.

### Temozolomide Administration

Temozolomide may be administered at the appropriate time, as determined by a medical practitioner. Preferably, it is administered beginning approximately 4 weeks following the completion of external beam radiotherapy. Subjects may commence temozolomide administration with a dosage regimen of about 150 mg/m²/day for 5 consecutive days every 28 days for up to 6 28-day cycles. Subjects who tolerate temozolomide at 150 mg/m²/dose without any attributable grade 3 or 4 toxicity may increase the temozolomide dose to 200 mg/m²/dose.

Criteria to be determined before initiation of temozolomide treatment include: WBC count; ANC count; platelet count; adequate hepatic function, including SGOT and bilirubin measurements; and adequate renal function, including creatinine level and/or creatinine clearance.

Temozolomide dosing levels may vary according to the level of temozolomide toxicity in each subject undergoing treatment. For example, the temozolomide dose may be adjusted as set forth below in Table 1:

TABLE 1

| Dose at Toxicity | | Modified Dose | |
|---|---|---|---|
| 75 | mg/m² (during XRT) | 60 | mg/m² |
| 60 | mg/m² (during XRT) | 50 | mg/m² |
| 200 | mg/m² | 150 | mg/m² |
| 150 | mg/m² | 125 | mg/m² |

### Example 4

### Results of a Phase II Study of 131-Iodine-Labeled Anti-Tenascin Murine Monoclonal Antibody 81C6 Administered to Deliver a Targeted Radiation Boost Dose of 44 Gy to the Surgically Created Cystic Resection Cavity Perimeter in the Treatment of Patients with Newly Diagnosed Primary and Metastatic Brain Tumors

Prior trials incorporating a “fixed” dose of ¹³¹I-labeled anti-tenascin monoclonal antibody 81C6 (¹³¹I-81C6) administered into the surgically created resection cavity (SCRC) of patients with either newly diagnosed or recurrent malignant glioma have been associated with encouraging survival and acceptable toxicity. In particular, previously performed Phase I and II trials incorporating a “fixed” dose of ¹³¹1-81C6) administered into the surgically created resection cavity (SCRC) of patients with newly diagnosed glioma reported 80 weeks and 79 weeks median survival, respectively.

Dosimetry analyses of patients treated on these studies predict that the delivery of a “targeted” 44 Gy boost to the SCRC by ¹³¹I-81C6 may be associated with a lower rate of toxicity and possibly improved overall outcome compared to the “fixed” dose regimen. The current study was designed to evaluate the efficacy and toxicity of administering a dose of ¹³¹I-81C6 antibody to achieve a “targeted” 44 Gy boost to the SCRC perimeter, in patients with newly diagnosed glioma.

### Materials and Method

Eligibility criteria include: adults with newly diagnosed and previously untreated malignant glioma; gross total resection; lack of communication between the resection cavity and the CSF space; KPS greater than 60%; and adequate bone marrow, kidney and hepatic function. A pretreatment dosimetry study with approximately 0.5 mCi of ¹³¹I-81C6 was performed to determine the therapeutic dose of ¹³¹I-81C6 required to achieve the 44 Gy “targeted” boost in each individual patient. Following the therapeutic dose of ¹³¹I-81C6 all patients underwent conventional external beam radiotherapy and systemic chemotherapy. Twenty-one patients were treated, including 15 with GBM and 6 with AA/AO (AA=anaplastic astrocytoma; AO=anaplastic oligodendroglioma). Of these, 20 received combination therapy with temozolomide. The median age was 49 years (range, 24-70) and 76% were male. The median dose of 131-I-81C6 administered was 62 mCi (range, 25-150).

### Results

Twenty patients successfully achieved a 44 Gy (+/−10%) boost to the SCRC perimeter. Toxicity was limited to grade 3 reversible hematologic toxicity in 15% of the subjects. No episodes of grade 4 toxicity occurred, and no episodes of delayed neurotoxicity have been documented. With a median follow-up of 62.7 weeks, the median survival for patients with newly diagnosed GBM is 93.9 weeks. This represents an approximately 15% increase in median survival reported previously in a Phase II clinical study involving patients with newly diagnosed malignant glioma. The median survival for the AA/AO patients has not been achieved. The administration of ¹³¹I-81C6 to achieve a 44 Gy “targeted” boost is feasible and associated with encouraging survival.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

1. A method of treating cancer in a subject in need thereof, said method comprising the steps of:
(a) administering to said subject a treatment effective amount of a therapeutic antibody; and
(b) administering to said subject a treatment effective amount of an alkylating agent.

2. The method of claim 1, wherein the cancer is a solid tumor-based cancer.

3. The method of claim 1, wherein the cancer is lymphoma.

4. The method of claim 1, wherein the cancer is brain cancer.

5. The method of claim 4, wherein the brain cancer is glioblastoma.

6. The method of claim 2, wherein the solid tumor expresses tenascin.

7. The method of claim 1, wherein said therapeutic antibody specifically binds tenascin.

8. The method of claim 1, wherein the therapeutic antibody is monoclonal antibody 81C6.

9. The method of claim 1, wherein the therapeutic antibody is mouse-human chimeric monoclonal antibody 81C6 (ch81C6).

10. The method of claim 1, wherein the therapeutic antibody is murine monoclonal antibody 81C6 (mu81C6).

11. The method of claim 1, wherein said therapeutic antibody is coupled to a radionuclide.

12. The method of claim 1, wherein the alkylating agent is temozolomide or an analog, pharmaceutically acceptable salt or prodrug thereof.

13. The method of claim 2, wherein at least a portion of said tumor is surgically removed prior to said administering step (a).

14. The method of claim 1, wherein said therapeutic antibody is administered by intracranial injection to the site of said tumor.

15. The method of claim 1, wherein said therapeutic antibody is administered by a single intracranial injection to the site of said tumor.

16. The method of claim 1, wherein said therapeutic antibody is administered in a dose of between about 40 to about 50 Gy.

17. The method of claim 12, wherein said temozolomide is administered orally.

18. The method of claim 12, wherein said temozolomide is administered in a cycle of daily doses for between about 3 to about 7 consecutive days at a daily dose of from between about 50 to about 300 mg/m²/day.

19. The method of claim 18, wherein said cycle is repeated every two to five weeks for a total of up to about 10 cycles.

20. The method of claim 1, further comprising the step of:
(c) administering said subject external beam radiotherapy to the site of said brain tumor.

21. The method of claim 20, wherein said external beam radiotherapy is administered at a total dose of between about 30 to about 60 Gy to the site of said brain tumor.

22. The method of claim 1, wherein said antibody is monoclonal antibody 81C6 or an antibody that binds to the epitope bound by monoclonal antibody 81C6.

23. The method of claim 11, wherein said radionuclide is selected from the group consisting of ²²⁷Ac, ²¹¹At, ¹³¹Ba, ⁷⁷Br, ¹⁰⁹Cd, ⁵¹Cr, ⁶⁷Cu, ¹⁶⁵Dy, ¹⁵⁵⁵Eu, ¹⁵³Gd, ¹⁹⁸Au, ¹⁶⁶Ho, ^{113m}In, ^{115m}In, ¹²³I, ¹²⁵I, ¹³¹I, ¹⁸⁹Ir, ¹⁹¹Ir, ¹⁹²Ir, ¹⁹⁴Ir, ⁵²Fe, ⁵⁵Fe, ⁵⁹Fe, ¹⁷⁷Lu, ¹⁰⁹Pd, ³²P, ²²⁶Ra, ¹⁸⁶Re, ¹⁸⁸Re, ¹⁵³Sm, ⁴⁶Sc, ⁴⁷Sc, ⁷²Se, .⁷⁵Se, ¹⁰⁵Ag, ⁸⁹Sr, ³⁵S, ¹⁷⁷Ta, ^{117m}Sn, ¹²¹Sn, ¹⁶⁶Yb, ¹⁶⁹Yb, ⁹⁰Y, ²¹²Bi, ¹¹⁹Sb, ¹⁹⁷Hg, ⁹⁷Ru, ¹⁰⁰Pd, ^{101m}Rh, ²¹²Pb, ⁶⁴Cu, ²²⁵Ac, ²¹³Bi and ¹²⁴I.

24. The method of claim 1, wherein said therapeutic antibody is monoclonal antibody 81C6, and wherein said alkylating agent is temozolomide or an analog, pharmaceutically acceptable salt or prodrug thereof.

25. (canceled)

26. (canceled)

27. (canceled)

28. (canceled)
